(19) United States
(12) Patent Application Publication
(10) Pub. No.: US 2019/0346517 A1
(43) Pub. Date: Nov. 14, 2019

(54) MODULAR MAGNETIC RESONANCE IMAGING PROTECTION SYTEM

(71) Applicant: KONINKLIJKE PHILIPS N.V., EINDHOVEN (NL)

(72) Inventors: GEORGE RANDALL DUENSING, GAINESVILLE, FL (US); OLLI TAPIO FRIMAN, GAINSVILLE, FL (US); EZRA PETRUS ANTONIUS VAN LANEN, Niskayuna, NY (US); TRACY ALLYN WYNN, GAINESVILLE, FL (US)

(21) Appl. No.: 16/474,163

(22) PCT Filed: Dec. 26, 2017
(86) PCT No.: PCT/IB2017/058398

§ 371 Date: Jun. 27, 2019

Related U.S. Application Data
(60) Provisional application No. 62/442,489, filed on Jan. 5, 2017.

(51) Int. Cl. G01R 33/28; G01R 33/30; G01R 33/422; A61B 5/055

(52) U.S. Cl. CPC G01R 33/288; G01R 33/307; A61B 2560/0443; A61B 5/0555; G01R 33/422

(57) ABSTRACT

A modular magnetic resonance imaging protection system includes a support, a first platform and a second platform. The support passes through a bore of a magnetic resonance imaging system and includes a first guidance system. The first platform and second platform are each configured to support a patient. The first platform and second platform can each be guided from a carrier to the support through an acoustic shield. The first platform and second platform respectively include a second guidance system and a third guidance system to cooperatively guide the first platform and second platform along the support, into the bore of the magnetic resonance imaging system, and out of the bore of the magnetic resonance imaging system in cooperation with the first guidance system.

### BACKGROUND

Rooms that enclose magnetic resonance imaging (MRI) systems typically include radio frequency (RF) screening mechanisms in/on the walls and doors, such that the RF screening mechanisms define the perimeters of the rooms. The RF screening mechanisms are designed to enclose the MRI systems, to allow storage of MRI set-up accessories such as local RF coils, pads, interventional equipment, sheets and more in the rooms and RF screening mechanisms. The room interiors are not used during MRI operations for anything other than the MRI operations, and the doors to the rooms are kept closed, which in turn prevents free access during the MRI operations. MRI set-up accessories within the rooms cannot be used to prepare the next patients when the previous patients are still being examined in the MRI operations, and this results in relatively low efficiency for the use of the MRI systems. This is especially true for in-patient facilities which require transportation of the patients to and from bores of the MRI systems. That is, the overall MRI operations are slow and cumbersome because the next patients cannot be set up while the current patients are being imaged since the doors to the screened rooms stay closed and the MRI set-up accessories are located within the screened rooms.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a plan view of a room with a modular magnetic resonance imaging protection system, in accordance with a representative embodiment of the present disclosure.

FIG. 2A is a plan view of a modular magnetic resonance imaging protection system, in accordance with a representative embodiment of the present disclosure.

FIG. 2B is a cross-sectional view of a radio frequency shielding screen that includes a window, in accordance with a representative embodiment of the present disclosure.

FIG. 3 is another plan view of a room with a modular magnetic resonance imaging protection system, in accordance with a representative embodiment of the present disclosure.

FIG. 4 is another plan view of a room with a modular magnetic resonance imaging protection system, in accordance with a representative embodiment of the present disclosure.

FIG. 5 is a view of a process for managing patients using the modular magnetic resonance imaging protection system, in accordance with a representative embodiment of the present disclosure.

FIG. 6 is a plan view of a support and platform with guidance systems in a modular magnetic resonance imaging protection system, in accordance with a representative embodiment of the present disclosure.

FIG. 7 is another plan view of a room with a modular magnetic resonance imaging protection system, in accordance with a representative embodiment of the present disclosure.

FIG. 8A is another plan view of a modular magnetic resonance imaging protection system, in accordance with a representative embodiment of the present disclosure.

FIG. 8B is another cross-sectional view of a radio frequency shielding screen that includes a window, in accordance with a representative embodiment of the present disclosure.

FIG. 9 is another cross-sectional view of a radio frequency shielding screen that includes a window, in accordance with a representative embodiment of the present disclosure.

FIG. 10A is a cross-sectional view of guidance systems in a modular magnetic resonance imaging protection system, in accordance with a representative embodiment of the present disclosure.

FIG. 10B is another cross-sectional view of guidance systems in a modular magnetic resonance imaging protection system, in accordance with a representative embodiment of the present disclosure.

### DETAILED DESCRIPTION

In the following detailed description, for purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only, and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In view of the foregoing, the present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

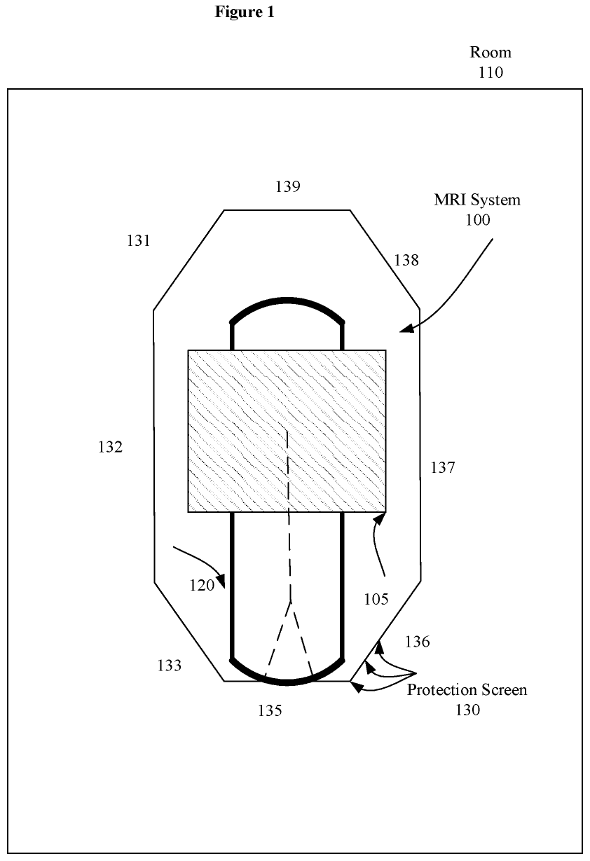

FIG. 1 is a plan view of a room with a modular magnetic resonance imaging protection system, in accordance with a representative embodiment of the present disclosure. In FIG. 1, a room 110 is an enclosure defined by walls, a floor, and a roof. Since FIG. 1 is a plan view, only four sides of the room 110 are outlined. In FIG. 1, the room 110 may, but does not necessarily, include radio frequency (RF) screening mechanisms in/on the walls and doors. A room 110 may be at a health care facility such as a hospital or outpatient center. As shown in FIG. 1, the magnetic resonance imaging system 100 is separated from (interior to) the walls of the room 110 at every point or position or location. The room 110 may be initially built or later adapted to enclose a magnetic resonance imaging (MRI) system 100 as shown.

The magnetic resonance imaging system 100 may be a conventional magnetic resonance imaging system 100. A magnetic resonance imaging system 100 is a complex system that uses magnets to align and realign hydrogen nuclei (protons) in water molecules in a subject (e.g., human) being imaged. Strong magnetic fields are applied to align and realign the proton “spins”. Magnetic resonance imaging system 100 can include radio frequency (RF) coils to then selectively deliver a B1 field in a transmit stage. In a receive stage, the hydrogen atoms return to an original position (i.e., the position before the selective delivery of the B1 field) and emanate a weak radio frequency signal which can be picked up and used to produce images. The room 110 may be initially built or later adapted specifically to contain the strong magnetic fields while also repelling radio frequency signals from the outside.

The magnetic resonance imaging system 100 includes a cylindrical magnet 105 with a bore. The cylindrical magnet 105 may be housed in a housing that serves as an exterior surface, but is otherwise the outermost functional element around the bore. Other elements of the magnetic resonance imaging system 100 may be arranged in the bore of the cylindrical magnet 105 or exterior surface Additionally, the patient (not shown) being imaged is placed in the bore of the cylindrical magnet 105. The noted cylindrical magnet 105 is only an example, as other types of magnets can be used for magnetic resonance imaging systems, including a split cylindrical magnet or an open magnet. Other elements arranged in the bore of the cylindrical magnet 105 may include a cylindrical body coil immediately interior to the cylindrical magnet 105, wherein the body coil provides the main uniform, static, magnetic field that excites and aligns the hydrogen atoms. Examples of uniform magnetic fields provided by a body coil are 1.5 Teslas, 3.0 Teslas, or 7.0 Teslas. The magnetic resonance imaging system 100 in FIG. 1 may also include field gradient coils immediately interior to the body coil, wherein the field gradient coils are relative low frequency coils used to choose a plane of interest relative to X, Y and Z axes. Such field gradient coils are used to generate a magnetic field across the subject in the bore of the cylindrical magnet 105 by using a field gradient. Finally, the magnetic resonance imaging system 100 in FIG. 1 may also include a radio frequency coil immediately interior to the field gradient coil. The radio frequency coil is used to deliver the B1 field to selected slices of an imaging zone, to result in the delivery of the B1 field is to manipulate orientations of magnetic spins of hydrogen nuclei within the imaging zone. The radio frequency coil is used in the transmit stage, and may be used in some systems for the receive stage. That is, once a transmit cycle is complete, the hydrogen atoms return to original positions, and emanate a weak radio frequency signal. This weak radio frequency signal from the hydrogen atoms returned to their original positions is what is picked up in the receive stage of the magnetic resonance imaging cycle.

In FIG. 1, a support 120 passes through the bore of the magnetic resonance imaging system 100. A first guidance system is shown by the broken line on the support 120 in FIG. 1. For example, the first guidance system may be a track, a rut or a rail fixture. The support 120 may provide both the first guidance system and, e.g., a table, a support bed or other form of support for the first guidance system. Alternatively, the support 120 may include only the first guidance system, such as when the first guidance system is a rigid and structurally sound rail system that can support the weight of a platform that carries a patient. As another example, the first guidance system on the support 120 may be either a male or female member of a male/female pair.

In FIG. 1, a protection screen 130 forms a perimeter around the cylindrical magnet 105 and the support 120. The protection screen 130 may be an acoustic shield. The protection screen 130 may include eight modular components (e.g., panels) 131, 132, 133, 135, 136, 137, 138, 139 fastened together. The eight modular components 131, 132, 133, 135, 136, 137, 138, 139 may be of different shapes and widths, and serve as a substantial physical barrier fastened to (e.g., installed in) the room 110 and to each other. The eight modular components 131, 132, 133, 135, 136, 137, 138, 139 may be pre-fabricated prior to delivery for installation in the room 110. For example, the eight modular components 131, 132, 133, 135, 136, 137, 138, 139 may be pre-fabricated aluminum panels. Moreover, the eight modular components 131, 132, 133, 135, 136, 137, 138, 139 may each be encased in an exterior fixture to provide a pleasing aesthetic appearance. Such an exterior fixture may include drywall, tile, or other forms of recognizable wall materials, and the eight modular components 131, 132, 133, 135, 136, 137, 138, 139 may be encased in such exterior fixtures prior to delivery for installation in the room 110.

As an example, the eight modular components 131, 132, 133, 135, 136, 137, 138, 139 are shown to have linear profiles in FIG. 1. However, one or more of the eight modular components 131, 132, 133, 135, 136, 137, 138, 139 may have a curved profile.

Additionally, the eight modular components 131, 132, 133, 135, 136, 137, 138, 139 may be fastened together by screws, nails, adhesives, welding, or other fasteners. A result of the fastening of the eight modular components 131, 132, 133, 135, 136, 137, 138, 139 may be that the protection screen 130 has no, or substantially no, gaps or holes other than windows and doors provided intentionally as described herein. That is, the protection screen 130 may serve as, and appear as, a single integrated barrier to incoming and outgoing light and electromagnetic signals. As noted, the protection screen 130 may be an acoustic shield.

The protection screen 130 prevents radio frequency signals from entering the interior of the perimeter formed by the protection screen 130. The protection screen 130 also helps protect the exterior of the perimeter and items or people in the exterior from the magnetic/electromagnetic fields created by the cylindrical magnet 105 and other elements (e.g., cylindrical body coil, field gradient coils, radio frequency coil) interior to the cylindrical magnet 105. Additionally, the protection screen 130 physically prevents items outside the perimeter from being drawn towards and even into the bore of the cylindrical magnet 105 by the magnetic/electromagnetic fields created by the cylindrical magnet 105 and other elements (e.g., cylindrical body coil, field gradient coils, radio frequency coil) interior to the cylindrical magnet 105.

The protection screen 130 defines a RF screen room. Using multiple platforms for loading and unloading patients to the support 120 outside of the RF screen room, it is possible to decrease the time the magnetic resonance imaging system 100 is not running, and increase the number of patients that can be scanned, for example, in a period such as 1 hour or 4 hours. Furthermore, the protection screen 130 can be assembled from modular components such that the RF screen rooms can be standardized. Non-ambulatory patients can therefore be exchanged using the modular magnetic resonance imaging protection system described herein as quickly as walking patients can be exchanged.

Moreover, patient preparation that occurs while the magnetic resonance imaging system 100 is operational for another patient can be kept quiet outside the shielded room as the protection screen 130 provides both acoustic attenuation and electromagnetic attenuation. This effect can be useful compared to, for example, a double door system that permits access in and out of the perimeter of the protection screen 130 without loss of the electromagnetic screening function and set up to take place within the RF room.

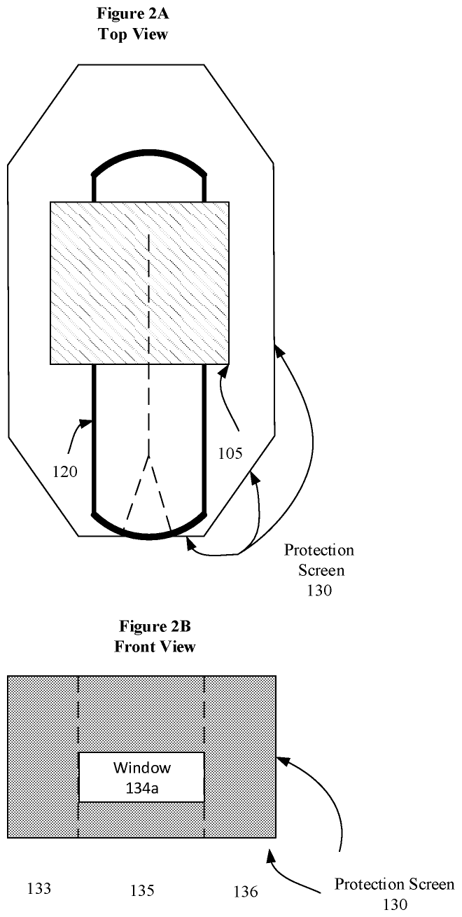

FIG. 2A is a plan view of a modular magnetic resonance imaging protection system, in accordance with a representative embodiment of the present disclosure. In FIG. 2A, the protection screen 130 and the elements of the magnetic resonance imaging system 100 (i.e., the cylindrical magnet 105 and the support 120) are shown in isolation.

FIG. 2B is a cross-sectional view of a radio frequency shielding screen that includes a window, in accordance with a representative embodiment of the present disclosure. In FIG. 2B, the protection screen 130 is shown from a front (cross-sectional) view. In the front (cross-sectional) view, the protection screen 130 includes three of the modular components 133, 135, 136. The middle modular component 135 has a window 134a. The window 134a has a lower edge above the bottom of the modular component 135, and an upper edge below the top of the modular component 135. Although the window 134a is shown extending from one side of the modular component 135 to the other, the window 134a may extend across a smaller cross-section of the modular component 135. In an embodiment, the height of the lower edge of the window 134a is approximately the same, substantially the same, or identical to the height of an upper surface of the support 120. Additionally, the window 134a may have a limited aperture size, for example, in the order of 1 square meter. Using the window 134a, the patient enters the perimeter of the protection screen 130 via the window at the appropriate height for entry into the core of the cylindrical magnet 105. The window 134a into the perimeter of the protection screen 130 allows the support 120 top to pass through at a location at least approximately 2.5 meters from the isocenter of the cylindrical magnet 105. The window 134a may be a divided double window with one portion used for passage through the protection screen 130 in one direction (e.g., in) and one portion used for passage through the protection screen 130 in another direction (e.g., out).

Additionally, a lower edge of the window 134a may include a guidance system compatible with the guidance system of the support 120. For example, a rail or rails may be installed on or embedded in a lower edge of the window 134a.

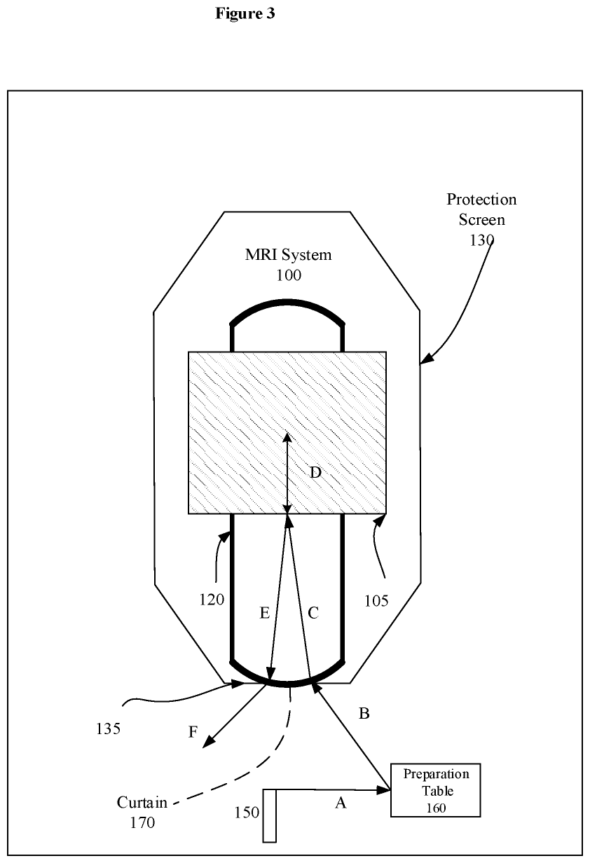

FIG. 3 is another plan view of a room with a modular magnetic resonance imaging protection system, in accordance with a representative embodiment of the present disclosure. In FIG. 3, a (first) platform 150 is shown outside of the perimeter formed by the protection screen 130. The first platform 150 may be a modified stretcher, bed, gurney, sled, or other form of furniture that can support a patient in a prone position. The first platform 150 may be carried on a carriage so that a medical professional can maneuver the carriage and first platform 150 around inside the room 110 outside of the perimeter of the protection screen 130. Additionally, the first platform 150 may have a guidance system that works with a guidance system on the support 120 to guide the first platform 150 along the support 120. The guidance systems of the first platform 150 and support 120 can cooperatively guide the first platform 150 into the bore of the cylindrical magnet 105 of the magnetic resonance imaging system 100. The guidance systems of the first platform 150 and support 120 can also cooperatively guide the first platform 150 out of the bore of the cylindrical magnet 105 of the magnetic resonance imaging system 100.

Cooperative guidance as described herein may include the guidance system on the support 120 being fixed while the guidance system of the first platform 150 moves. As a result, the first platform 150 may move or be moved relative to the support 120. The guidance system of the first platform 150 may, for example, be inserted into and move along the guidance system of the support 120. The first platform 150 may be moved manually, by a power source external to the protection screen 130 that mechanically conveys power through the protection screen 130 to the support 120, by gravity, and/or by any other known mechanism for compatibly moving an item near an magnetic resonance system 100.

Additionally, the guidance system of the first platform 150 may be held relatively in or to the guidance system of the support 120 by, for example, gravity. Alternatively, or in addition, the guidance system of the first platform 150 may be held relatively in or to the guidance system of the support 120 by a mechanical arrangement, such as by a portion of the guidance system of the support 120 being above a portion of the guidance system of the first platform 150.

In FIG. 3, a curtain 170 is shown as a form of barrier extending from the front of the modular component 135 at the front of the protection screen 130. The curtain 170 can serve as a visual barrier between the first platform 150 (and a patient on the first platform 150) and a second platform 180 to be shown and explained in subsequent figures.

In FIG. 3, a preparation table 160 is also shown outside of the perimeter formed by the protection screen 130. The preparation table 160 carries supplies for patients before they are subject to imaging by the magnetic resonance imaging system 100. A process for patients using the magnetic resonance imaging system 100 can be explained using the path of arrows labelled A, B, C, D, E and F in FIG. 3.

At process A in FIG. 3, the first platform 150 is moved from, for example a room entrance, to the preparation table 160. The first platform 150 may be moved using, for example, a carriage or gurney. At the preparation table 160, a patient on the first platform 150 is prepared for imaging and, for example, one or more coils are applied to (e.g., placed on) the patient.

At process B in FIG. 3, the first platform 150 is moved from the preparation table 160 to the modular component 135 at the front of the protection screen 130. At the front of the modular component 135, a battery pack may be applied to the coil(s) applied to the patient. Subsequently, the first platform 150 and patient move through the window 134a of the modular component 135. A bottom of the window 134a may include a guidance system (e.g., a rail) compatible with the guidance system of the support 120. For example, guidance systems on the first platform 150 and the support 120 may be matched, placed in contact, aligned etc. as the first platform 150 is moved to the support 120.

At process C in FIG. 3, the first platform 150 is moved along the support 120 to the front of the cylindrical magnet 105 of the magnetic resonance imaging system 100. The first platform 150 may be moved manually using the guidance systems of the first platform 150 and the support 120, or may be moved automatically using the guidance systems of the first platform 150 and the support 120. For example, a motor placed external to the protection screen 130 may automatically pull a conveyor of the support 120 to move the first platform 150 to the front of the magnetic resonance imaging system 100. Such motor and/or conveyor (not shown) may be additional guidance components that supplement the guidance systems of the first platform 150 and the support 120.

At process D in FIG. 3, the first platform 150 is moved into the bore of the cylindrical magnet 105, and the patient is subject to imaging. Here is an appropriate place to note that the materials used for the first platform 150, support 120 and guidance systems of the first platform 150 and support 120 should be of types consistent with materials used for existing conventional supports. Such materials specifically include plastics, and specifically exclude ferrous metallic compounds.

At process E in FIG. 3, the first platform 150 is moved along the support 120 from the front of the cylindrical magnet 105 of the magnetic resonance imaging system 100 to the window 134a of the modular component 135. At process F, the first platform 150 is moved from the window 134a out of the perimeter formed by the protection screen 130. As seen in FIG. 3, when the first platform 150 is moved out of the window 134a at F in FIG. 3, the first platform 150 is visually separated from the starting point of the first platform 15 at A in FIG. 3.

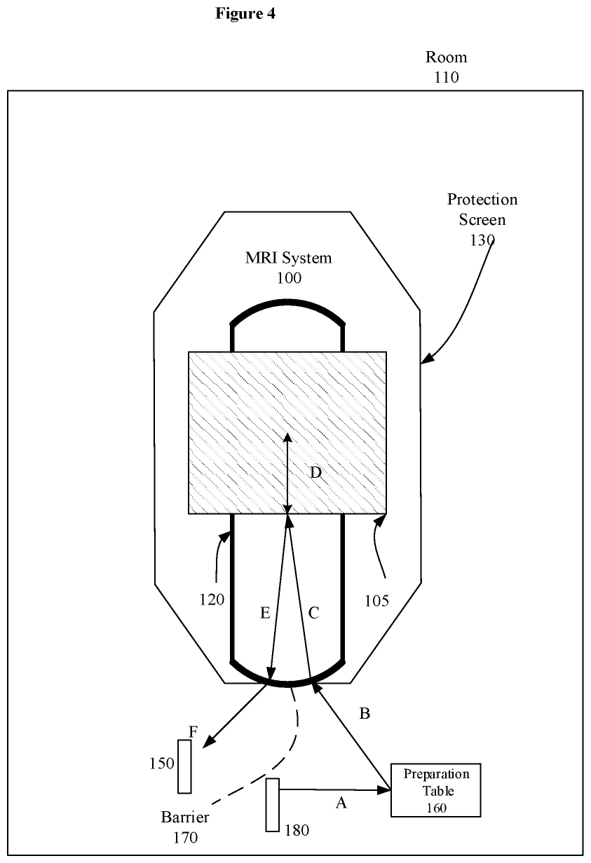

FIG. 4 is another plan view of a room with a modular magnetic resonance imaging protection system, in accordance with a representative embodiment of the present disclosure. In FIG. 4, a second platform 180 is introduced at process A. Processes A, B, C, D, E and F in FIG. 4 are identical to the processes labeled the same in FIG. 3, but are performed for a patient on the second platform 180 instead of the patient on the first platform 150. As shown, the area in the room 110 outside of the perimeter of the protection screen 130 can be used for processing patients both before and after using the magnetic resonance imaging system 100.

Given the additional room for the preparation in room 110 (i.e., outside of the protection screen 130), and the use of the barrier 171, two different patients can be simultaneously processed using the first platform 150 and second platform 180. In other words, one patient can be prepared to enter the magnetic resonance imaging system 100 as another patient is in the magnetic resonance imaging system 100 or as or immediately after the other patient has exited the magnetic resonance imaging system.

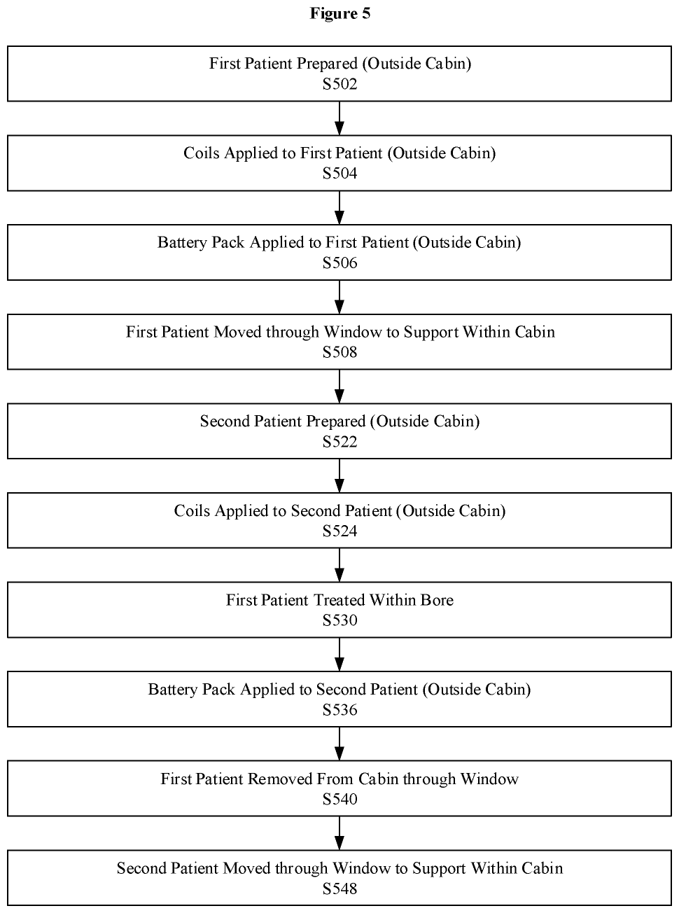

FIG. 5 is a view of a process for managing patients using the modular magnetic resonance imaging protection system, in accordance with a representative embodiment of the present disclosure. The process in FIG. 5 is like the processes explained already with respect to FIGS. 3 and 4. At S502, a first patient is prepared outside of the cabin formed by the protection screen 130. At S504, coils are applied to the first patient outside the cabin. At S506, a battery pack is applied to the first patient outside the cabin. At S580, the first patient is moved through the window 134a to the support 120 within the cabin.

At S522, a second patient is prepared outside the cabin. At S524, coils are applied to the second patient outside the cabin. At S530, the first patient on the first platform 150 is treated within the bore. At S536, a battery pack is applied to a second patient outside the cabin. At S540, the first patient is removed from the cabin through the window 134a. At S548, the second patient is moved through the window 134a to the support 120 in the cabin.

As should be evident, in FIG. 5 patients on different platforms 150 and 180 can be processed simultaneously in the room 110. The order of several steps shown in FIG. 5 is arbitrary and can be readily varied. The process in FIG. 5 is intended to show that the second patient can be prepared (e.g., at S522) as soon as the first patient is moved through the window 134a to the support 120 within the cabin. The process in FIG. 5 is also intended to show that the second patient can be moved through the window 134a to the support 120 within the cabin as soon as the first patient is removed from the cabin through the window 134a.

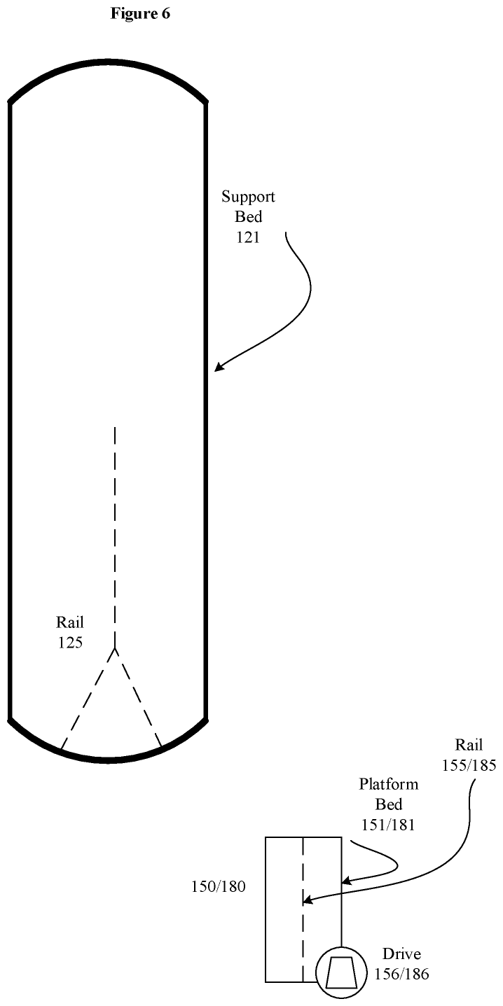

FIG. 6 is a plan view of a support 120 and first platform 150/second platform 180 with guidance systems in a modular magnetic resonance imaging protection system, in accordance with a representative embodiment of the present disclosure. In FIG. 6, the support 120 is shown with a rail 125. The rail 125 can be installed on, placed on, affixed to, attached to, overlaid on, embedded in and/or disposed on a bed 121 of the support 120. The first platform 150 and/or second platform 180 in FIG. 6 is shown with a rail 155/185, as well as with an affiliated drive 156/186. The rail 155/185 can be installed on, placed on, affixed to, attached to, overlaid on, embedded in and/or disposed on a bed 151/181 of the first platform 150/second platform 180.

The drive 156/186 can include, for example, a motor (stator/rotor) and a conveyor system such as with a durable plastic chain. The motor can be placed outside of the perimeter of the protection screen 130, and can pull the conveyor system to move the first platform 150/second platform 180 using the rail 125 and rail 155/185. Thus, the first platform 150/second platform 180 can be moved into the bore of the cylindrical magnet 105, and then out of the bore of the cylindrical magnet 105. As an alternative to the drive 156/186, a conventional power source can be used, such as by plugging a conveyor into an electrical outlet outside of the perimeter formed by the protection screen 130.

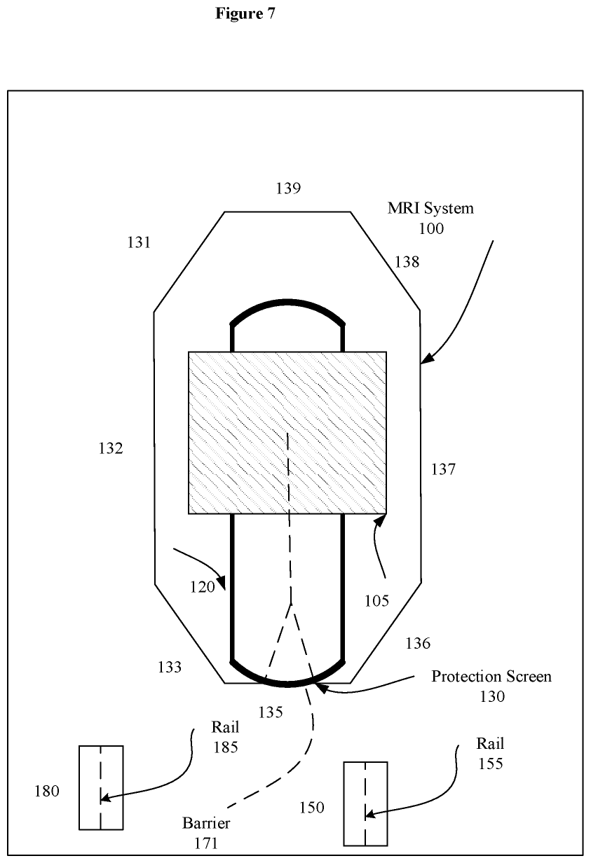

FIG. 7 is another plan view of a room with a modular magnetic resonance imaging protection system, in accordance with a representative embodiment of the present disclosure. In FIG. 7, the first platform 150 and second platform 180 are shown on separate sides of the barrier 171. As should be evident, the barrier 171 may be a curtain, but may also be a permanent or temporary wall, a movable wall, or another form of visual barrier. Additionally, the first platform 150 in FIG. 7 is shown specifically with a rail 1S5, and the second platform 180 is shown specifically with a rail 185. The rails 1S5 and 185 may be placed into contact in at least one dimension with a rail on the support 120. For example, the rails on the first platform 150/second platform 180 may be one of male and female, and the rail on the support 120 may be the other of male and female. A variety of alternative configurations for rails 155/185 are possible, including interlocking rails.

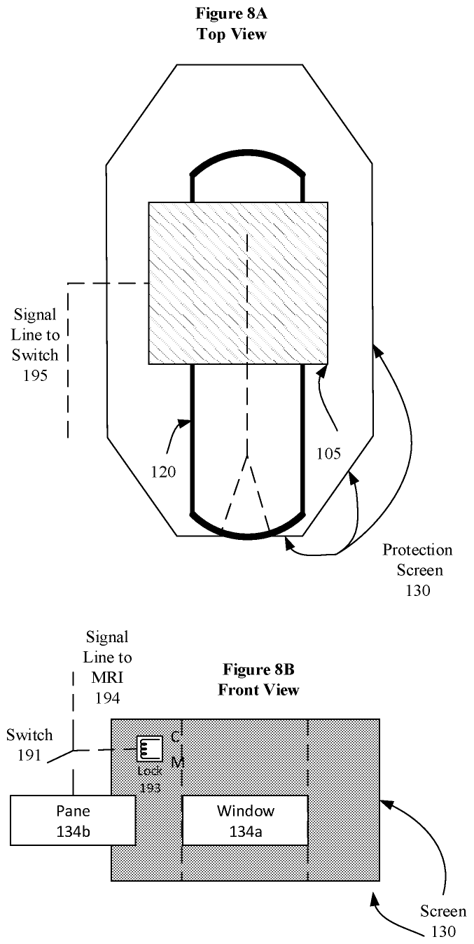

FIG. 8A is another plan view of a modular magnetic resonance imaging protection system, in accordance with a representative embodiment of the present disclosure. In FIG. 8A, a signal line 195 to a switch is shown leading out from the cylindrical magnet 105. The signal line 195 may be used to carry a signal indicating when the cylindrical magnet 105 is operating/operational. The signal line 195 is used together with the signal line 194 to the magnetic resonance imaging system 100 in FIG. 8B.

FIG. 8B is another cross-sectional view of a radio frequency shielding screen that includes a window, in accordance with a representative embodiment of the present disclosure. In FIG. 8B, a signal line 194 to the magnetic resonance imaging system 100 may be used to carry a signal indicating when the window 134a is open or closed. A switch 191 in FIG. 8B may be used to switch off the magnetic resonance imaging system 100 when the window 134a is unexpectedly opened when the magnetic resonance imaging system 100 is operating.

In FIG. 8B, a lock 193 may be an electronic lock to electronically lock the window 134a close while the magnetic resonance imaging system 100 is operating. The lock 193 may be an alternative to the switch 191 and signal line 194 to the magnetic resonance imaging system, or the lock 193 may be a supplement to the switch 191, or the switch 191 may be a supplement to the lock 193.

In FIG. 8B, a pane 134 is shown adjacent to the window 134a. The pane 134b may be the same material as the modular component 135. For example, the pane 134b and the modular component 135 may both be aluminum. The position of the pane 134b may be used by the switch 191 to identify the proper signal to place on the signal line 194 to the magnetic resonance imaging system 100. For example, when the pane 134b covers the window 134a, a signal may be sent indicating the operation of the magnetic resonance imaging system 100 is safe. When the pane 134b does not cover the window 134a, a signal may be sent indicating the operation of the magnetic resonance imaging system 100 is unsafe and should be stopped.

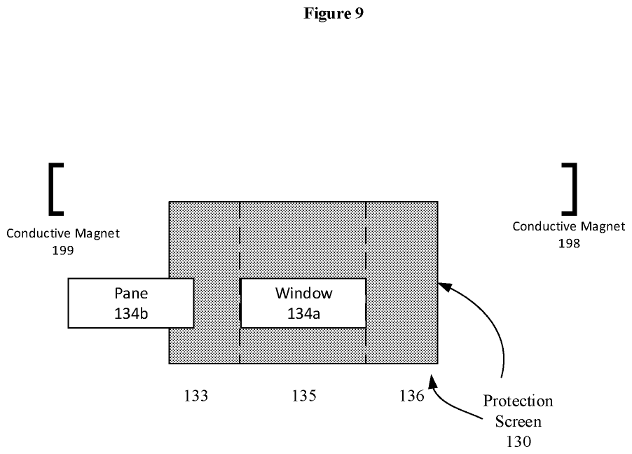

FIG. 9 is another cross-sectional view of a radio frequency shielding screen that includes a window, in accordance with a representative embodiment of the present disclosure. In FIG. 9, conductive magnets 198 and 199 are placed on either side of the modular components 133, 135 and 136 of the protection screen 130. The conductive magnets 198 and 199 may be used as supplemental protection mechanisms when the window 134a is open. The conductive magnets 198 and 199 may draw items to the side of the window 134a when the window 134a is open. Additionally, the conductive magnets 198 and 199 may be selectively turned on, for example using the switch 191 in FIG. 8, only when the window 134a is not covered by the pane 134b.

For a 3T Ingenia magnet, the force on soft iron is approximately 0.5 g (acceleration of gravity) at 2.5 meters. The magnetic field is approximately 0.014T while the gradient is approximately 0.029 T/m at 2.5 meters. Force on a ferrous material is proportional to both the field and the gradient of the field. The area outside the perimeter of the protection screen 130 is a controlled space due to the proximity to the magnetic resonance imaging system 100; however, the presence of ferro-magnetic materials in the area is still possible in the event of, for example, an oversight in screening. The conductive magnets 198 and 199 address the admittedly low risk of the small ferrous objects being drawn through the open window 134a. By producing appropriate local magnetic field gradients, the already-low risk of entry of such materials through the window can be greatly reduced, thus increasing the safety the modular magnetic resonance imaging protection system described herein.

Because the local fields in the region around the window 134a and the exterior of the perimeter formed by the protection screen 130 are comparatively low, the use of normal conductive magnets 198, 199 is plausible. The conductive magnets 198, 199 produce a field and gradient that are on the order of or higher than the stray field existing at the location of the window 134a with reasonable power requirements. By careful shape of the local fields, most of the aperture of the window 134a can made safe from ferrous ballistic threats by pulling objects to the sides of the window 134a instead of through the window 134a. Alternatively, warning of incoming ferrous ballistic threats can be made by increasing the force from the side, and then decreasing the force again before entry to enable observation. That is, the force from the normal conductive magnets 198, 199 is limited relative to the force imposed by the electromagnetic fields of the magnetic resonance imaging system 100.

Additionally, as explained previously with respect to FIG. 8B, the window 134a can be provided with a switch 191 (e.g., an interlock) that enables current flow to produce the fields from the conductive magnets 198, 199 only when the window is open. Therefore, the magnetic resonance imaging system 100 is not operated when the extra field from the conductive magnets 198, 199 is active. As such, the conductive magnets 198, 199 and switch 191 may be considered a safety device that prevents a stray field from affecting magnetic resonance imaging, and when the window is closed the switch 191 ensures the conductive magnets 198, 199 present no hazard. Such a switch 191 also permits use of gradient amplifiers as current sources for the conductive magnets 198, 199 since the conductive magnets 198, 199 are used when the window is open. As previously explained, the gradient at 2.5 meters for the 3T shielded magnet is approximately the maximum gradient produced within the magnetic resonance imaging system 100 during operation.

In another embodiment alternative to the use of conductive magnets 198, 199, passive magnetic material can be provided within walls that include the protection screen 130 to affect the local field and gradients. Passive magnetic materials can be used when the materials are part of the overall system design, since these materials will always remain effective while the protection screen 130 stands.

Another alternative embodiment uses electromagnets below or above the patient that scan as the support 120 moves past. The electromagnets in this alternative embodiment are designed to attract small objects. Larger objects can be sensed by such electromagnets such that a pull imposed on the electromagnets by larger objects is detected by force sensors, and the detected pull is used to generate and send a signal to stop the movement of the support 120. This embodiment is an alternative to deflecting or otherwise drawing or pulling an object away from the support 120, and has the advantage of being closer to the support 120 surface rather than on, for example, walls external to the perimeter of the protection screen 130.

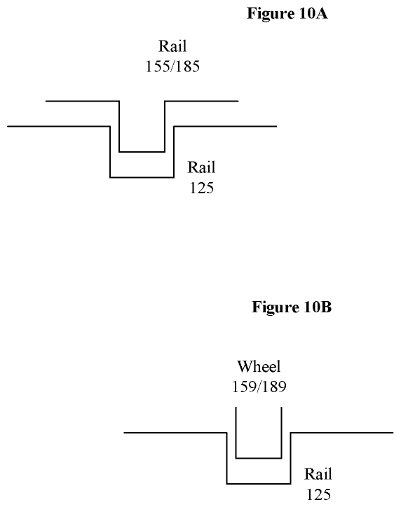

FIG. 10A is a cross-sectional view of guidance systems in a modular magnetic resonance imaging protection system, in accordance with a representative embodiment of the present disclosure. In FIG. 10A, rails 155/185 on the first platform 150/second platform 180 are male rails that align and can contact (female) rail 125 of the support 120.

FIG. 10B is another cross-sectional view of guidance systems in a modular magnetic resonance imaging protection system, in accordance with a representative embodiment of the present disclosure. In FIG. 10B, wheels 159/189 of the first platform 150/second platform 180 may be used to cooperatively guide the first platform 150/second platform 180 along a (female) rail 125 of the support 120.

According to an aspect of the present disclosure, a modular magnetic resonance imaging protection system includes a support that passes through a bore of a magnetic resonance imaging system. The support includes a first guidance system. The modular magnetic resonance imaging protection system also includes a first platform configured to support a patient. The first platform can be guided from a carrier to the support through an acoustic shield. The platform includes a second guidance system configured to cooperatively guide the first platform along the support, into the bore of the magnetic resonance imaging system, and out of the bore of the magnetic resonance imaging system in cooperation with the first guidance system. The modular magnetic resonance system further includes a second platform configured to support a patient. The second platform can be guided from the carrier to the support through the acoustic shield. The second platform includes a third guidance system configured to cooperatively guide the second platform along the support, into the bore of the magnetic resonance imaging system, and out of the bore of the magnetic resonance imaging system in cooperation with the first guidance system.

According to another aspect of the present disclosure, the first guidance system includes a first rail on the support. The second guidance system includes a second rail configured to contact the first rail in at least one dimension. The third guidance system includes a third rail configured to contact the first rail in at least one dimension.

According to still another aspect of the present disclosure, the support includes a first support top that includes a first portion of the first guidance system and a second support top that includes a second portion of the first guidance system. The first portion and the second portion merge together to pass through the bore.

According to yet another aspect of the present disclosure, the first guidance system includes a rail system.

According to another aspect of the present disclosure, the second rail is configured to be interchangeably interlocked with the first rail in the at least one dimension. The third rail is configured to be interchangeably interlocked with the first rail in the at least one dimension.

According to still another aspect of the present disclosure, the modular magnetic resonance imaging protection system also includes a drive that drives the first platform and the second platform along the first portion of the first guidance system to enter the bore via the first support top. The drive drives the first platform and the second platform along the second portion of the first guidance system to exit the bore and the second support top.

According to yet another aspect of the present disclosure, the modular magnetic resonance imaging protection system includes a barrier that visually separates a space that includes the first platform and the second platform so that the first platform is visually separated from the second platform.

According to another aspect of the present disclosure the modular magnetic resonance imaging protection system also includes a first radio frequency shielding screen fastened to a room that includes the support, the first platform and the second platform. A second radio frequency shielding screen is fastened to the first radio frequency shielding screen and to the room to form a protection screen. The second radio frequency shielding screen includes an opening through which the first platform and the second platform can be guided. The protection screen is installed within the room adjacent to and substantially forming a perimeter around the support and the magnetic resonance imaging system to separate the support and the magnetic resonance imaging system from the first platform and the second platform.

According to still another aspect of the present disclosure, the support passes through the bore substantially at a height through which the first platform and the second platform can be guided through the opening.

According to yet another aspect of the present disclosure, the modular magnetic resonance imaging protection system includes a movable pane that can be moved to open and close the opening. Additionally, in another aspect of the present disclosure, the opening is a window.

According to another aspect of the present disclosure, the protection screen is installed at least 2.5 meters from a center of a magnet of the magnetic resonance imaging system.

According to still another aspect of the present disclosure, the movable pane, the first radio frequency shielding screen and the second radio frequency shielding screen constructed of, made of, formed of, built of, and/or assembled of the same material.

According to yet another aspect of the present disclosure, the modular magnetic resonance imaging protection system includes a lock that only allows the movable pane to be moved from the window when the magnetic resonance imaging system is not in operation.

According to another aspect of the present disclosure the modular magnetic resonance imaging protection system includes a switch that switches the magnetic resonance imaging system off when the movable pane is moved from the window.

According to still another aspect of the present disclosure, the modular magnetic resonance imaging protection system includes conductors used to shape an electromagnetic field that draws ferrous objects from a location of the opening so as to prevent the ferrous objects from passing through the opening.

Accordingly, a standardized radiofrequency cabin can be formed using a protection screen 130, with a window 134a, and a dual table (platform) system which optimizes set up of next patients during an MRI exam of previous patients. Specifically, the radiofrequency cabin front wall (e.g., 135) is located just outside of the zone where ferrous objects could be pulled in. The force limitations imply that even if ferrous rolling objects are brought into this zone, they will not get pulled in, because the opening of the window 134a begins at a height from the floor, such as 2 feet above the floor. This operates as a stop, like a ship door.

Moreover, the support 120 can be designed with little or no electronics internally and with reliable simple docking (if needed) for any necessary power port or optical port. The guidance system on the support 120 allows two tracks or rails to converge and diverge, like a railroad system. When the support 120 is formed of two support tops, the support tops can support a merge into the single track or rail that enters the bore of the cylindrical magnet 105. This permits rapid removal of one patient, followed immediately by loading of a second patient that has been fully prepared for imaging. Any support mechanism/track may be used to move the patients. Advantageously, wireless coils can be used to provide complete set up on the support with no electronics within the bore or immediate area at all, which substantially increases patient throughput.

Additionally, as described herein, patient preparation is provided within the same room as a magnetic resonance imaging system 100, but outside of a perimeter provided by the protection screen 130. The area outside of the protection screen 130 is shielded from radio frequency signals from within, and the area within the protection screen 130 is shielded from radio frequency signals from outside. This permits full set up, including coils, of the patient while another scan is occurring, while also enabling a rapid exchange between the patients.

Although the modular magnetic resonance imaging protection system has been described regarding several exemplary embodiments, it is understood that the words that have been used are words of description and illustration, rather than words of limitation. Changes may be made within the purview of the appended claims, as presently stated and as amended, without departing from the scope and spirit of the modular magnetic resonance imaging protection system in its aspects. Although the modular magnetic resonance imaging protection system has been described regarding particular means, materials and embodiments, the modular magnetic resonance imaging protection system is not intended to be limited to the particulars disclosed; rather the modular magnetic resonance imaging protection system extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

According to an aspect of the present disclosure, a modular magnetic resonance imaging protection system includes a support that passes through a bore of a magnetic resonance imaging system. The support includes a first guidance system. The modular magnetic resonance imaging protection system also includes a first platform configured to support a patient. The first platform can be guided from a carrier to the support through an acoustic shield. The platform includes a second guidance system configured to cooperatively guide the first platform along the support, into the bore of the magnetic resonance imaging system, and out of the bore of the magnetic resonance imaging system in cooperation with the first guidance system. The modular magnetic resonance system further includes a second platform configured to support a patient. The second platform can be guided from the carrier to the support through the acoustic shield. The second platform includes a third guidance system configured to cooperatively guide the second platform along the support, into the bore of the magnetic resonance imaging system, and out of the bore of the magnetic resonance imaging system in cooperation with the first guidance system.

According to another aspect of the present disclosure, the first guidance system includes a first rail on the support. The second guidance system includes a second rail configured to contact the first rail in at least one dimension. The third guidance system includes a third rail configured to contact the first rail in at least one dimension.

According to still another aspect of the present disclosure, the support includes a first support top that includes a first portion of the first guidance system and a second support top that includes a second portion of the first guidance system. The first portion and the second portion merge together to pass through the bore.

According to yet another aspect of the present disclosure, the first guidance system includes a rail system.

According to another aspect of the present disclosure, the second rail is configured to be interchangeably interlocked with the first rail in the at least one dimension. The third rail is configured to be interchangeably interlocked with the first rail in the at least one dimension.

According to still another aspect of the present disclosure, the modular magnetic resonance imaging protection system also includes a drive that drives the first platform and the second platform along the first portion of the first guidance system to enter the bore via the first support top. The drive drives the first platform and the second platform along the second portion of the first guidance system to exit the bore and the second support top.

According to yet another aspect of the present disclosure, the modular magnetic resonance imaging protection system includes a barrier that visually separates a space that includes the first platform and the second platform so that the first platform is visually separated from the second platform.

According to another aspect of the present disclosure the modular magnetic resonance imaging protection system also includes a first radio frequency shielding screen fastened to a room that includes the support, the first platform and the second platform. A second radio frequency shielding screen is fastened to the first radio frequency shielding screen and to the room to form a protection screen. The second radio frequency shielding screen includes an opening through which the first platform and the second platform can be guided. The protection screen is installed within the room adjacent to and substantially forming a perimeter around the support and the magnetic resonance imaging system to separate the support and the magnetic resonance imaging system from the first platform and the second platform.

According to still another aspect of the present disclosure, the support passes through the bore substantially at a height through which the first platform and the second platform can be guided through the opening.

According to yet another aspect of the present disclosure, the modular magnetic resonance imaging protection system includes a movable pane that can be moved to open and close the opening. Additionally, in another aspect of the present disclosure, the opening is a window.

According to another aspect of the present disclosure, the protection screen is installed at least 2.5 meters from a center of a magnet of the magnetic resonance imaging system.

According to still another aspect of the present disclosure, the movable pane, the first radio frequency shielding screen and the second radio frequency shielding screen constructed of, made of, formed of, built of, and/or assembled of the same material.

According to yet another aspect of the present disclosure, the modular magnetic resonance imaging protection system includes a lock that only allows the movable pane to be moved from the window when the magnetic resonance imaging system is not in operation.

According to another aspect of the present disclosure the modular magnetic resonance imaging protection system includes a switch that switches the magnetic resonance imaging system off when the movable pane is moved from the window.

According to still another aspect of the present disclosure, the modular magnetic resonance imaging protection system includes conductors used to shape an electromagnetic field that draws ferrous objects from a location of the opening to prevent the ferrous objects from passing through the opening.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for streamlining the disclosure. This disclosure is not to be interpreted as reflecting an intention that the claimed embodiments require more features than are expressly recited in each claim. Rather, as the following claims reflect, inventive subject matter may be directed to less than all the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

1. A modular magnetic resonance imaging protection system, comprising:
an acoustic shield;
a support that passes through a bore of a magnetic resonance imaging system, the support comprising a first guidance system;
a first platform configured to support a patient, wherein the first platform is configured to be guided from a carrier to the support through the acoustic shield, the first platform comprising a second guidance system configured to cooperatively guide the first platform along the support, into the bore of the magnetic resonance imaging system, and out of the bore of the magnetic resonance imaging system in cooperation with the first guidance system; and
a second platform configured to support a patient, wherein the second platform is configured to be guided from the carrier to the support through the acoustic shield, the second platform comprising a third guidance system configured to cooperatively guide the second platform along the support, into the bore of the magnetic resonance imaging system, and out of the bore of the magnetic resonance imaging system in cooperation with the first guidance system.

2. The modular magnetic resonance imaging protection system of claim 1,
wherein the first guidance system comprises a first rail on the support,
the second guidance system comprises a second rail configured to contact the first rail in at least one dimension, and
the third guidance system comprises a third rail configured to contact the first rail in at least one dimension.

3. The modular magnetic resonance imaging protection system of claim 1,
wherein the support comprises a first support top that includes a first portion of the first guidance system and a second support top that includes a second portion of the first guidance system, and
the first portion and the second portion merge together to pass through the bore.

4. The modular magnetic resonance imaging protection system of claim 3, wherein the first guidance system comprises a rail system.

5. The modular magnetic resonance imaging protection system of claim 2,
wherein the second rail is configured to be interchangeably interlocked with the first rail in the at least one dimension, and
the third rail is configured to be interchangeably interlocked with the first rail in the at least one dimension.

6. The modular magnetic resonance imaging protection system of claim 3, further comprising:
a drive that drives the first platform and the second platform along the first portion of the first guidance system to enter the bore via the first support top, and that drives the first platform and the second platform along the second portion of the first guidance system to exit the bore and the second support top.

7. The modular magnetic resonance imaging protection system of claim 1, further comprising:
a barrier that visually separates a space that includes the first platform and the second platform so that the first platform is visually separated from the second platform.

8. The modular magnetic resonance imaging protection system of claim 1, further comprising:
a first radio frequency shielding screen as a first component of the acoustic shield, wherein the first radio frequency shielding screen is fastened to a room that includes the support, the first platform and the second platform; and
a second radio frequency shielding screen as a second component of the acoustic shield, wherein the second radio frequency shielding screen is fastened to the first radio frequency shielding screen and to the room to form a protection screen, and includes an opening through which the first platform and the second platform is configured to be guided,
wherein the protection screen is installed within the room adjacent to and substantially forming a perimeter around the support and the magnetic resonance imaging system to separate the support and the magnetic resonance imaging system from the first platform and the second platform.

9. The modular magnetic resonance imaging protection system of claim 8, wherein the support passes through the bore substantially at a height through which the first platform and the second platform can be guided through the opening.

10. The modular magnetic resonance imaging protection system of claim 8, further comprising:
a movable pane that is configured to be moved to open and close the opening,
wherein the opening comprises a window.

11. The modular magnetic resonance imaging protection system of claim 8, wherein the protection screen is installed at least 2.5 meters from a center of a magnet of the magnetic resonance imaging system.

12. The modular magnetic resonance imaging protection system of claim 10,
wherein the movable pane, the first radio frequency shielding screen and the second radio frequency shielding screen comprise the same material.

13. The modular magnetic resonance imaging protection system of claim 10, further comprising:
a lock that only allows the movable pane to be moved from the window when the magnetic resonance imaging system is not in operation.

14. The modular magnetic resonance imaging protection system of claim 10, further comprising:
a switch that switches the magnetic resonance imaging system off when the movable pane is moved from the window.

15. The modular magnetic resonance imaging protection system of claim 8, further comprising:
conductors used to shape an electromagnetic field that draws ferrous objects from a location of the opening to prevent the ferrous objects from passing through the opening.

16. A magnetic resonance imaging protection system, comprising:
a protection screen defining a radio frequency (RF) screen room enclosing a magnetic resonance imaging (MRI) system;
a radio frequency screen room enclosing the protection screen, there being a perimeter space extending between the protection screen and the radio frequency screen room.

17. The magnetic resonance imaging protection system of claim 16 wherein the protection screen comprises a plurality of modular panels which are fastened together.

18. The magnetic resonance imaging protection system of claim 16, wherein the protection screen includes a window sized to pass a patient and having a lower edge that is above a bottom of the protection screen and an upper edge that is below a top of the protection screen.

19. The magnetic imaging protection system of claim 18 further comprising:
conductive magnets arranged at sides of the window.
